(19) United States
(12) Patent Application Publication
(10) Pub. No.: US 2021/0009950 A1
(43) Pub. Date: Jan. 14, 2021

(54) ANGIOGENESIS-INDUCING METHOD USING NEURAL STEM CELL

(71) Applicant: MEDINNO INC., Gyeonggi-do (KR)

(72) Inventors: Kyeung Min Joo, Seoul (KR); Hyun Nam, Gyeonggi-do (KR)

(21) Appl. No.: 16/980,886

(22) PCT Filed: Jan. 30, 2019
(86) PCT No.: PCT/KR2019/001287

§ 371 Date: Sep. 15, 2020

(30) Foreign Application Priority Data
Mar. 16, 2018 (KR) 10-2018-0031099
Jan. 29, 2019 (KR) 10-2019-0011224

(51) Int. Cl. C12N 5/0797; A61K 35/30; A61K 9/00; A61K 35/44; A61P 9/00

(52) U.S. Cl. CPC C12N 5/0623; A61K 35/30; A61K 9/0019; C12N 2500/20; A61P 9/00; C12N 2500/02; A61K 35/44

(57) ABSTRACT

The present invention relates to an angiogenesis-inducing method using neural stem cells. According to the present invention, neural stem cells derived from human brain tissues release MCP-1 or Gro, thus exhibiting an angiogenesis-inducing effect. When CoCl₂ was treated to the neural stem cells of the present invention and when the neural stem cells were cultured in a hypoxia chamber, it was observed that a hypoxia condition increased the release amounts of MCP-1 and Gro. Therefore, the human brain tissue-derived neural stem cells of the present invention can be used to induce angiogenesis.

### TECHNICAL FIELD

The present invention relates to an angiogenesis-inducing method using neural stem cells.

### BACKGROUND ART

Stem cells are capable of self-renewal and differentiation into various cells, can be derived from an embryo or fetus. It is also known that resident stem cells in adult tissues can regenerate their own tissues. Mesenchymal stem cells are well known as adult stem cells and are the leading material for clinical researches worldwide.

Neural stem cells are stem cells that are important in treatment of degenerative neurological disorders because they are capable of self-proliferation and differentiation into neurons, astrocytes or oligodendrocytes. Neural stem cells were first identified in rodent, mice, and are known to exist in a specific region of the brain.

Since then, it has been known that neural stem cells are present in the human brain, and human neural stem cells acquired from fetal brain tissue can be expanded ex vivo and have been used in clinical trials. Recently, it has been reported that neural stem cells exist in adult human brain tissue, and they can be preferably expanded by adherent culture, rather than sphere culture.

Angiogenesis refers to the growth of a new vessel, and a process mainly dependent on migration, proliferation, and formation of capillary endothelial cells. Angiogenesis may form a blood vessel under physiological and pathological conditions including wound healing or cancer. The regulation of angiogenesis is site- and stimulus-dependent, and may, in each case, involve a unique combination of regulatory molecules. During angiogenesis, endothelial cells rapidly proliferate, escaping from their stationary state.

Meanwhile, Matrigel (trade name for the product by BD Bioscience) is a protein complex extracted from Engelbreth-Holm-Swarm (EHS) mouse sarcoma cells, and contains extracellular matrix (ECM) components such as laminin, collagen and heparan sulfate proteoglycan, and growth factors such as a fibroblast growth factor (FGF), an epidermal growth factor (EFG), an insulin-like growth factor (IGF), a transforming growth factor-beta (TGF-β) and a platelet-derived growth factor (PDGF). The complex constituting Matrigel is used as a cell culture matrix by providing a complicated extracellular environment found in various tissues.

Matrigel has been used to reinforce cardiomyocytes and endothelial cells transplanted into an ischemic animal model, and since embryonic stem cells (ESCs) have self-renewability and pluripotency, Matrigel has been used for ex vivo culture of murine and human embryonic stem cells.

Therefore, the inventors confirmed from an experiment using Matrigel in which, among stem cells with commercial usefulness in medical substances and cosmetics having various functions, neural stem cells derived from human brain tissue secrete MCP-1 and Gro to have an angiogenesis-inducing effect, and thus the present invention was completed.

### DISCLOSURE

### Technical Problem

As a result of the inventors conducting a study on functions of conventional neural stem cells, other than a known function, such as differentiation into neural cells, it was confirmed that neural stem cells derived from human brain tissue secrete MCP-1 and Gro, thereby obtaining an angiogenesis-inducing effect, and based on this finding, the present invention was completed.

Therefore, the present invention is directed to providing an angiogenesis-inducing method, which includes:

(a) culturing neural stem cells under a hypoxia condition; and

(b) injecting the cultured neural stem cells into a subject.

However, technical problems to be solved in the present invention are not limited to the above-described problems, and other problems which are not described herein will be fully understood by those of ordinary skill in the art from the following descriptions.

### Technical Solution

To attain the purpose of the present invention, the present invention provides an angiogenesis-inducing method, which includes:

(a) culturing neural stem cells under a hypoxia condition; and

(b) injecting the cultured neural stem cells into a subject.

In one embodiment of the present invention, the neural stem cells may be derived from human brain tissue.

In another embodiment of the present invention, the step (a) may include treating neural stem cells with CoCl₂ or culturing the neural stem cells in a hypoxia chamber.

In still another embodiment of the present invention, the step (b) may include injecting vascular endothelial cells along with the neural stem cells into a subject.

In yet another embodiment of the present invention, the cultured neural stem cells may secrete MCP-1 or Gro.

In yet another embodiment of the present invention, the culture of the neural stem cells in a hypoxia chamber may be performed for 12 to 72 hours.

In yet another embodiment of the present invention, the CoCl₂ may have a concentration of 100 to 500 μM.

### Advantageous Effects

It was confirmed that neural stem cells derived from human brain tissue according to the present invention have an effect of forming blood vessels by secreting MCP-1 or Gro, and when CoCl₂ is applied to the neural stem cells of the present invention and the neural stem cells are cultured in a hypoxia chamber, a hypoxia condition induces neural stem cells to increase secretion of MCP-1 and Gro, and thus it is expected that angiogenesis can be induced using human brain tissue-derived neural stem cells of the present invention.

### DESCRIPTION OF DRAWINGS

FIGS. 1A to 1C show the angiogenesis-inducing effect of neural stem cells through a Matrigel plug assay according to one embodiment of the present invention, wherein FIG. 1A is a diagram showing the outline of a Matrigel plug assay, FIG. 1B is a diagram showing the presence or absence of angiogenesis on day 3 after transplantation of a mixture of neural stem cells and vascular endothelial cells which is discerned by color due to penetration of red blood cells, and FIG. 1C is a diagram showing the histologic result of the Matrigel plug assay by H&E staining.

FIGS. 2A and 2B show qualitative and quantitative analysis of angiogenesis-related factors of neural stem cells using a cytokine array (FIG. 2A) and enzyme-linked immunosorbent assay (ELISA, FIG. 2B) according to one embodiment of the present invention.

FIG. 3 shows the angiogenesis inhibitory effect by MCP-1 and Gro-neutralizing antibodies according to one embodiment of the present invention.

FIG. 4 shows a signaling pathway in which angiogenesis-related expression of MCP-1 and Gro secreted under a hypoxia condition induced by CoCl₂ treatment in neural stem cells according to one embodiment of the present invention is increased.

FIG. 5 is a diagram illustrating an experimental design for confirming the level of MCP-1 and Groα secreted from neural stem cells, following induction of a hypoxia condition by treating CoCl₂ with various concentrations.

FIG. 6A shows results obtained by measuring the concentration of MCP-1 by ELISA following treatment of CoCl₂ with various concentrations.

FIG. 6B shows results obtained by measuring Groα concentrations by ELISA following treatment of CoCl₂ with various concentrations.

FIG. 7 is a diagram illustrating an experimental design for confirming the expression of MCP-1 and Groα secreted from neural stem cells following induction of a hypoxia condition by culturing the neural stem cells in a hypoxia chamber.

FIG. 8 shows the results obtained by measuring the concentrations of MCP-1 and Groα over time by ELISA following culture of neural stem cells in a hypoxia chamber.

### MODES OF THE INVENTION

The present invention provides an angiogenesis-inducing method, which includes:

(a) culturing neural stem cells under a hypoxia condition; and

(b) injecting the cultured neural stem cells into a subject.

The neural stem cells of the present invention may be derived from human brain tissue, but the present invention is not limited thereto.

According to the present invention, the subject refers to a target in need of induction of angiogenesis, and more specifically, a mammal such as a human or a non-human primate, a mouse, a rat, a dog, a cat, a horse, or a cow.

According to the present invention, the hypoxia condition may be a cell culture condition having an oxygen partial pressure of 1% to 5%, but the present invention is not limited thereto.

According to the present invention, the step (a) may include treating neural stem cells with CoCl₂ or culturing the neural stem cells in a hypoxia chamber, but the present invention is not limited thereto.

According to the present invention, the step (b) may include injecting vascular endothelial cells along with the neural stem cells into a subject.

According to the present invention, when the neural stem cells are cultured in a hypoxia chamber, the hypoxia condition may be induced to increase the secretion amounts of MCP-1 and Gro, and the culture may be performed for 12 to 72 hours.

According to an exemplary embodiment of the present invention, when the cells are cultured for 48 to 72 hours, it is preferable because MCP-1 and Groα expression levels are increased, but the culture period is not limited thereto.

In addition, the cultured neural stem cells may induce formation of blood vessels by secreting MCP-1 or Gro, but the present invention is not limited thereto.

Here, the “monocyte chemoattractant protein-1 (MCP-1)” is a monocytic chemotactic factor and a protein of the chemokine β subfamily. MCP-1 has a potent chemotactic effect on monocytes, and exhibits effects on T lymphocytes, mast cells and basophilic leukocytes. Although MCP-1 is made by various types of cells (leukocytes, platelets, fibroblasts, endothelial cells and smooth muscle cells), it has highest specificity for monocytes and macrophages, and constitutes an activating stimulus as well as a chemotactic factor, resulting in induction of processes of forming multiple inflammatory factors (superoxides, arachidonic acid and derivatives, cytokines/chemokines) and amplification of phagocytosis.

In addition, the “growth-related oncogene (Gro)” is also referred to as melanoma growth stimulatory activity (MGSA), and includes three types of isoforms such as GROα (MGSAα, CXCL1), GROβ (MGSAβ, CXCL2) and GROγ (MGSAγ, CXCL3), which belong to CXC chemokines. The Gro includes its first two cysteine residues having a CXC structure and a glutamic acid-leucine-arginine (ELR) motif.

When CoCl₂ is applied to neural stem cells, a hypoxia condition may be induced and result in the increase of MCP-1 and Gro secretion, and in this case, the CoCl₂ concentration may be 100 to 500 μM. According to an exemplary embodiment of the present invention, the neural stem cells treated with CoCl₂ at a concentration of 500 μM for 24 hours or 100 μM and for 24 hours and then further cultured for 24 hours after replacement of the medium, are preferable because MCP-1 and Gro secretion are high, but the present invention is not limited to the concentration.

In one embodiment of the present invention, through histological analysis using a Matrigel plug assay and H&E staining, the angiogenesis-inducing effect of neural stem cells was confirmed (see Examples 1 and 2).

In another embodiment of the present invention, the amounts of MCP-1 and Gro secreted from neural stem cells were confirmed using a cytokine assay and ELISA (see Example 3).

In still another embodiment of the present invention, the angiogenesis-inducing effect by MCP-1 and Gro was confirmed using a neutralizing antibody against MCP-1 and Gro, respectively (see Example 4).

In yet another embodiment of the present invention, increase in MCP-1 and Groα expression levels by CoCl₂ in neural stem cells were confirmed (see Example 5).

In yet another embodiment of the present invention, MCP-1 and Groα expression levels after neural stem cells are cultured in a hypoxia chamber were confirmed (see Example 6).

Hereinafter, to help in understanding the present invention, exemplary examples will be suggested. However, the following examples are merely provided to understand the present invention more easily, and not to limit the present invention.

### EXAMPLE 1

### Matrigel Plug Assay

Experiments for verifying angiogenesis capability were performed with experimental groups consisted of a Matrigel-only group, a human umbilical cord vein epithelial cell (HUVEC)-only group, an adult human multipotent neural cell (ahMNC)-only group, a fetal neural stem cell (fNSC) and HUVEC mixed group, and an ahMNC and HUVEC-mixed group.

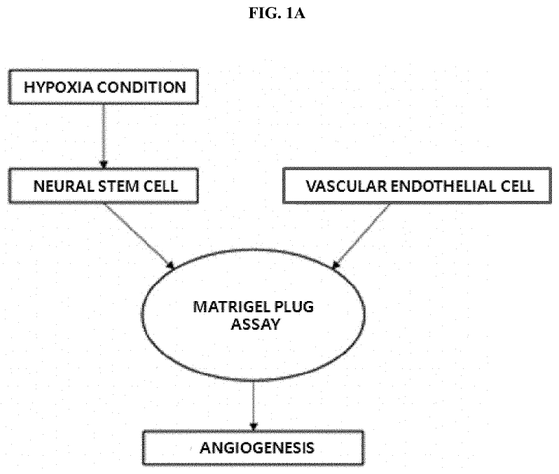

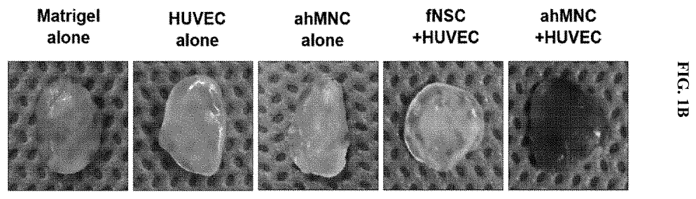

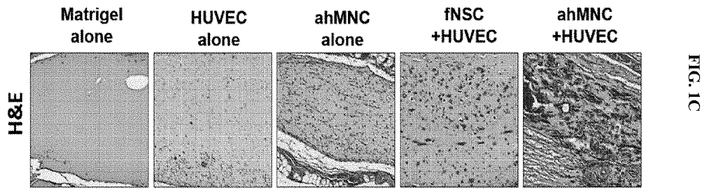

Neural stem cells (ahMNCs or a negative control fNSCs) and/or HUVECs were mixed with 200 μL of Matrigel at a 1:1 ratio such that a total cell number became 2×10⁶, and then transplanted subcutaneously into immunodeficient mice using a syringe. On day 3 after transplantation, the transplanted Matrigel was separated and then the presence or absence of blood vessel formation was discerned by color due to penetration of red blood cells. As a result, as shown in FIG. 1B, the ahMNC and HUVEC-mixed group was shown red.

Accordingly, from this result, blood vessels are formed by co-transplantation of neural stem cells and vascular endothelial cells.

### EXAMPLE 2

### Histological Analysis

The Matrigel plugs of Example 1 were fixed with 4% PFA, and blocks was made. For H&E staining, 5-μm-thick tissue sections were made, placed on a slide, and then subjected to deparaffination, followed by H&E staining.

As a result, as shown in FIG. 1C, in the ahMNC and HUVEC-mixed group, the formation of blood vessels was confirmed.

Accordingly, from this result, it was seen that blood vessels are formed by co-transplantation of neural stem cells and vascular endothelial cells.

### EXAMPLE 3

### Quantification of MCP-1 and Gro using Cytokine Array and ELISA

To confirm the angiogenesis-inducing effect of neural stem cells, growth factors or cytokines secreted from neural stem cells were qualitatively and quantitatively analyzed.

Three different types of neural stem cells such as NS14-001TL, NS14-008TL and NS14-015TL were seeded in 100-mm dishes, cultured for 3 to 4 days to be approximately 70% confluency, washed twice with phosphate buffered saline (PBS), and then cultured in the DMEM/F12 medium for 24 hours. Afterward, a supernatant (conditioned medium) was harvested. A cytokine array was performed using a human cytokine array (RayBio Human Cytokine Antibody Array C Series 1000).

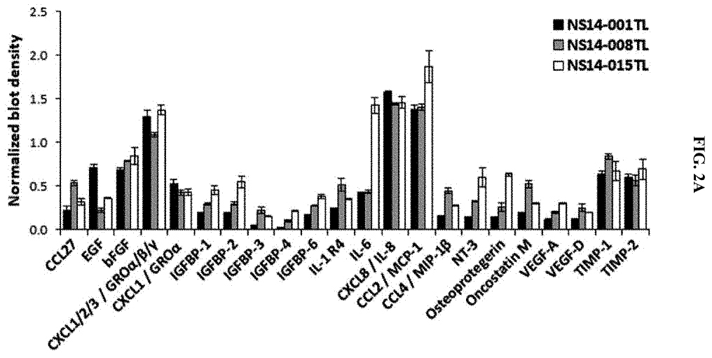

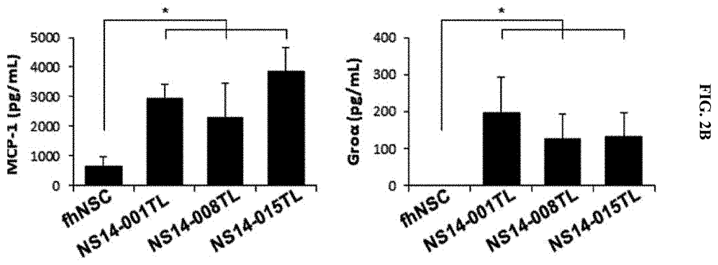

As a result, as shown in FIG. 2A, it was confirmed that various types of growth factors or cytokines such as MCP-1, Gro and IL-8 were secreted.

In addition, for quantification of MCP-1 and Gro found by the cytokine array, ELISA was performed with Quantikine ELISA kits (R&D Systems) using the recovered conditioned medium of the three different types of neural stem cells such as NS14-001TL, NS14-008TL and NS14-015TL.

As a result, as shown in FIG. 2B, compared with a control, fNSC, the amounts of MCP-1 and Groα were increased in all of the three types of neural stem cells.

### EXAMPLE 4

### Confirmation of Angiogenesis-Inducing Effect of MCP-1 and Gro

To confirm the angiogenesis-inducing effect of MCP-1 and Gro, a neutralizing antibody against each of MCP-1 and Gro was mixed with Matrigel and then transplanted to confirm angiogenesis inhibition.

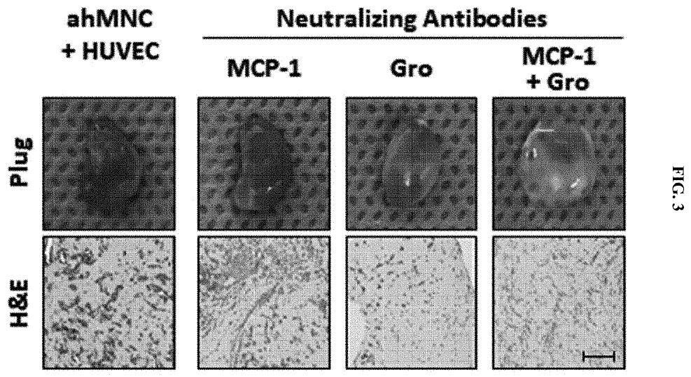

As a result, as shown in FIG. 3, when antibodies blocking the functions of MCP-1 and Gro were transplanted with Matrigel plugs, there was little angiogenesis. Moreover, when the antibodies blocking the functions of MCP-1 and Gro were mixed, it was confirmed that a synergistic angiogenesis inhibitory effect was exhibited.

### EXAMPLE 5

### Confirmation of MCP-1 and Gro Expression Levels by CoCl₂ Treatment in Neural Stem Cells

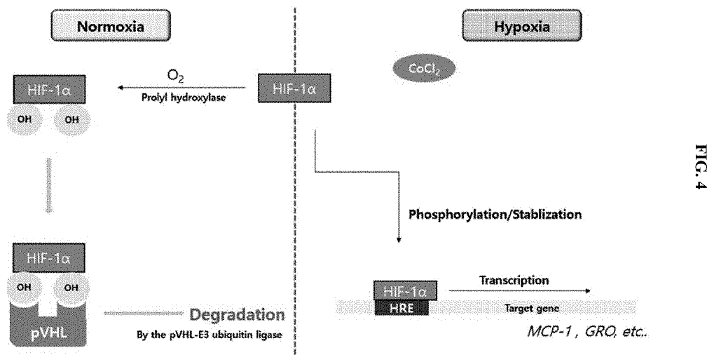

As shown in FIG. 4, when CoCl₂ was treated at various concentrations such as 100, 200, 300, 400 and 500 μM to reflect a hypoxia condition, the expression of MCP-1 and Gro increased by stabilizing HIF-1α, a transcription factor.

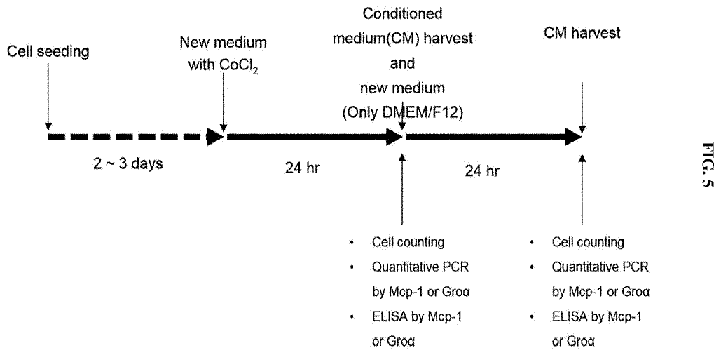

5-1. CoCl₂ treatment in neural stem cells Human adult neural stem cells were cultured in a 100-mm culture dish for 2 to 3 days to reach approximately 70 to 80% confluency. Afterward, to reflect a hypoxia condition, CoCl₂ was treated at various concentrations, for example, 100, 200, 300, 400 and 500 μM, and after 24 hours, the conditioned medium was harvested and replaced with DMEM/F12. After 24 hours, the conditioned medium was harvested and then analyzed. This process is schematically shown in FIG. 5.

5-2. Quantification of MCP-1 and Groα using ELISA For the quantification of MCP-1 and Groα in the conditioned medium recovered in Example 5-1, ELISA was performed using the Quantikine ELISA kits (R&D Systems).

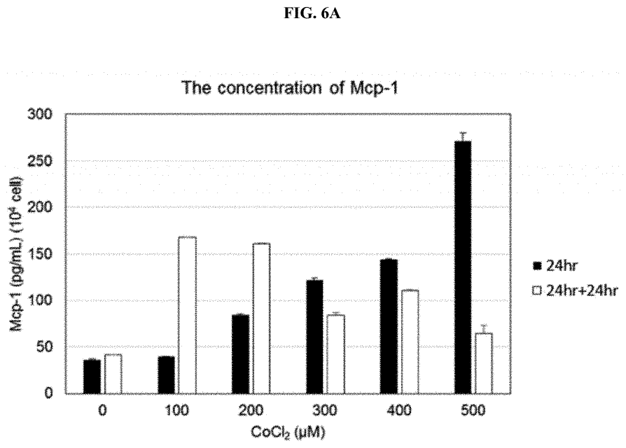

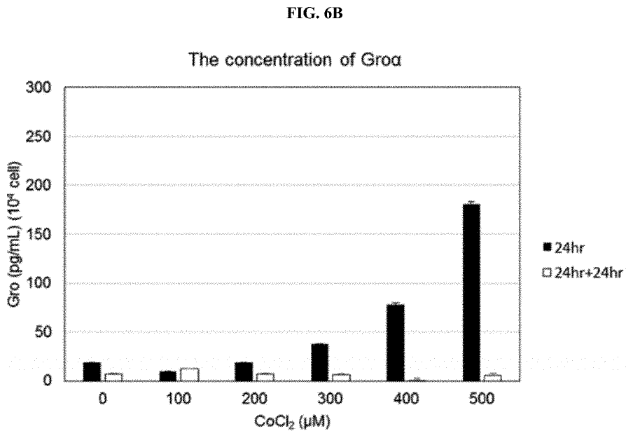

As a result, as shown in FIG. 6A, the amount of MCP-1 at 24 hours after CoCl₂ treatment, increased in proportion to the concentration of the treated CoCl₂. However, in the result of another 24 hours after the medium was replaced with DMEM/F12, the expression level of MCP-1 was highest in the case of treatment of 100 μM CoCl₂.

When the CoCl₂ concentration was 300 μM or more, compared to treatment at 100 and 200 μM, the amount of MCP-1 was reduced.

In addition, as shown in FIG. 6B, while the amount of Groα, at 24 hours after CoCl₂ treatment, increased in a concentration-dependent manner. The highest was observed when the CoCl₂ concentration was 500 μM. In the result of another 24 hours after the medium was replaced with DMEM/F12, it was confirmed that the amount of Groα was highest when 100 μM CoCl₂ was treated, but the Groα amount was reduced when the CoCl₂ concentration was 200 μM or more, compared to 100 μM.

### Example 6

### Confirmation of MCP-1 and Groα Expression Levels in Neural Stem Cells Cultured in Hypoxia Chamber

6-1. Culture of Neural Stem Cells in Hypoxia Chamber

To induce a hypoxia condition of neural stem cells, neural stem cells were cultured in a hypoxia chamber for 12, 24, 48 or 72 hours, and then a conditioned medium was recovered. The amounts of secreted MCP-1 and Groα were measured.

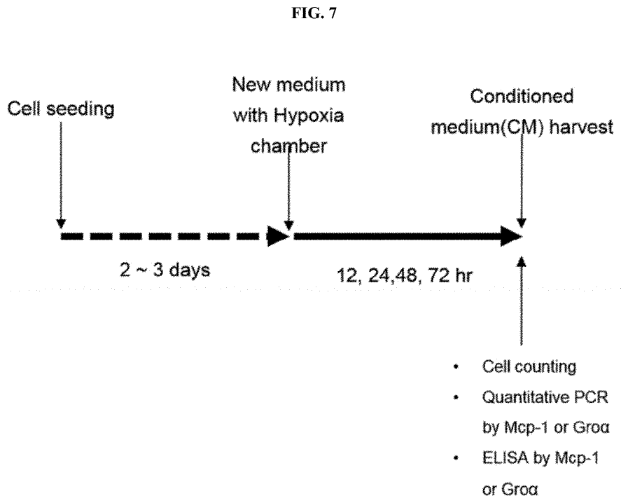

Human adult neural stem cells were cultured in a 100-mm culture dish to be 70 to 80% confluency for 2 to 3 days. Afterward, following replacement with a fresh medium, the cells were cultured in a hypoxia chamber for 12, 24, 48 or 72 hours, and then conditioned medium was recovered to perform ELISA. This procedure is schematically shown in FIG. 7.

6-2. Quantification of MCP-1 and Groα using ELISA

For quantification of MCP-1 and Groα in the conditioned medium recovered in Example 6-1, ELISA was performed using the Quantikine ELISA kits (R&D Systems).

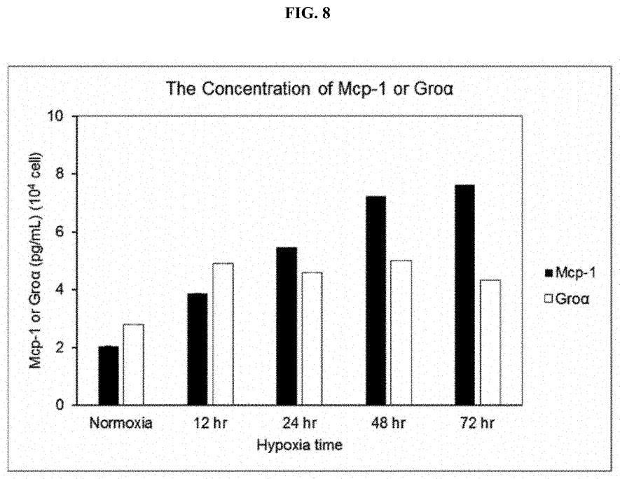

As a result, as shown in FIG. 8, while the expression of MCP-1 increased as the culture time passed, it was confirmed that the expression of Groα increased compared with a control, and remained almost constant after 48 hours in a hypoxic chamber.

It should be understood by those of ordinary skill in the art that the above description of the present invention is exemplary, and the exemplary embodiments disclosed herein can be easily modified into other specific forms without departing from the technical spirit or essential features of the present invention. Therefore, the exemplary embodiments described above should be interpreted as illustrative and not limited in any aspect.

### INDUSTRIAL APPLICABILITY

When CoCl₂ is treated to neural stem cells of the present invention and when the neural stem cells were cultured in a hypoxia chamber, it was confirmed that a hypoxia condition increases the amount of MCP-1 and Gro secretion. Human brain tissue-derived neural stem cells according to the present invention are cultured under a hypoxia condition so that they may be used to induce angiogenesis, and proteins or enzymes secreted from the neural stem cells may be used in angiogenesis-related developments such as purification of a conditioned medium and development of exosome. In addition, neural stem cells of the present invention may be used as neural stem cell therapeutics against degenerative neurological diseases such as stroke, spinal cord injury or Alzheimer's disease, for which angiogenesis is known to be a recovery mechanism, and are also expected to be used in development of therapeutic agents utilizing neural stem cell and their by-products against various diseases for which angiogenesis is known to be a recovery mechanism, other than degenerative neurological diseases.

1. An angiogenesis-inducing method, comprising:
(a) culturing neural stem cells under a hypoxia condition; and
(b) injecting the cultured neural stem cells into a subject.

2. The method of claim 1, wherein the neural stem cells are derived from human brain tissue.

3. The method of claim 1, wherein the step (a) includes treating neural stem cells with cobalt (II) chloride (CoCl2) or culturing neural stem cells in a hypoxia chamber.

4. The method of claim 1, wherein the step (b) includes injecting vascular endothelial cells along with the neural stem cells into a subject.

5. The method of claim 1, wherein the cultured neural stem cells secrete monocyte chemoattractant protein-1 (MCP-1) or a growth-related oncogene (Gro).

6. The method of claim 3, wherein the culturing the neural stem cells in a hypoxia chamber is performed for 12 to 72 hours.

7. The method of claim 3, wherein the CoCl₂ is treated at a concentration of 100 to 500 μM.
